(19) United States
(12) Patent Application Publication
(10) Pub. No.: US 2011/0009855 A1
(43) Pub. Date: Jan. 13, 2011

(54) EFFECT OF RADIOFREQUENCY FIELDS ON CELL GROWTH AND METHODS FOR LAPAROSCOPIC TREATMENT

(76) Inventors: Jean GOGUSEV, Paris (FR); Jean Bouquet de Joliniere, Suresnes (FR)

Correspondence Address: WILEY REIN LLP, 1776 K. STREET N.W., WASHINGTON, DC, 20006, US

(21) Appl. No.: 12/778,833

(22) Filed: May 12, 2010

Related U.S. Application Data
(63) Continuation of application No. PCT/EP2008/065620, filed on Nov. 14, 2008.
(60) Provisional application No. 60/996,377, filed on Nov. 14, 2007.

(51) Int. Cl. A61B 18/18; C12Q 1/02; C12Q 1/68

(52) U.S. Cl. 606/33; 435/29; 435/6

(57) ABSTRACT

The invention relates to the use of a radiofrequency/microwave treatment device and its effect on cell growth and survival, and in a more particular embodiment on endometriotic cells. In another aspect, the invention relates to the use of radiofrequency/microwave energy to effect cell growth in a desired population while substantially avoiding the deleterious heating and destructive effects on neighboring or nearby cells. Using the devices, specific treatment regimens and controlling energy output to cells and tissue can effect specific cells and diseases of humans, for example, those involving uncontrolled or deleterious cell growth.

This application claims priority benefit to U.S. Provisional application No. 60/996,377, filed Nov. 14, 2007, and International PCT application number PCT/EP2008/065620, filed Nov. 14, 2008, the entire contents of each are incorporated herein by reference.

### FIELD OF THE INVENTION

The invention relates to devices and methods for the treatment of tissue and the effect of energy transfer on cells. In one particular aspect, the invention relates to the use of a radiofrequency/microwave treatment device and its effect on cell growth and survival, and in a more particular embodiment on endometriotic cells. In another aspect, the invention relates to the use of radiofrequency/microwave energy to effect cell growth in a desired population while substantially avoiding the deleterious heating and destructive effects on neighboring or nearby cells. Thus, in one aspect the invention relates to the control of the energy treatment to cells and tissue. As shown below, short, focused treatments on cells result in a significant decrease in the proliferation rate of the cells and/or a degradation in cellular DNA. Based upon the effect on certain cells and tissues, a treatment can be designed or controlled to avoid or substantially avoid thermal destruction of surrounding cells. The device and methods of treatment can be used in a number of disease conditions, including, without limitation, endometriosis.

### RELEVANCE OF THE INVENTION AND DESCRIPTION OF RELATED ART

Various methods exists for the induction of toxic effects on live cells through exposure to radiofrequency (RF) energy. For the most part, the RF fields used in these treatments heat the cells, eventually resulting in cell death or damage. For several reasons, including heat transfer between cells and within tissue, the effect is generally over a relatively large area and not limited to specific cells.

As one type of cell proliferation disease, endometriosis is a common medical condition characterized by growth of tissue like endometrium, the lining of the uterus, beyond or outside the uterus. The disease affects an estimated 90 million women (usually around 30 to 40 years of age who have never been pregnant) around the world. In other rare cases, endometriosis has also been found in skin, lung, eye, diaphragm, and brain tissue.

Treatment is typically via surgery, and alternatives, especially laparoscopic or minimally invasive methods, are desired. However, typical RF devices and treatments are not specific, have side effects, and do not employ a controlled effect on cell growth.

### BRIEF SUMMARY OF THE INVENTION

The invention, in one aspect, satisfies the need for improved methods for treating proliferating or damaged cells. In other aspects, the methods can be used to cause cell damage and induce repair in a specific set of cells or tissue. Thus, a variety of cell treatment methods and regimens can be included in the scope of the invention. In part, these treatments are based upon the recognition of certain effects on the treated cells, effects that can be controlled and limited in size and extent within tissue. In another aspect, the methods of the invention relate to measuring the effect of radiofrequency/microwave treatments on cells and tissue. In another aspect, the invention relates to treatments that decrease cell growth or proliferation.

More particularly, the present findings indicate that treatment of endometriotic cells, stromal cells, and fibroblasts by radiofrequency/microwave (RF/EMFs) fields inhibits their proliferation and colony-forming capacity. Accordingly, RF/MW treatment can be an important tool and method for a variety of cell and tissue treating methods, including laparoscopic treatment of endometriotic lesions, treatment of aged or damaged skin, and similar treatments of humans and animals.

Throughout this disclosure, applicants refer to journal articles, patent documents, published references, web pages, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples that follow are merely exemplary of the scope of this invention and content of this disclosure and do not limit the scope of the invention. In fact, one skilled in the art can devise and construct numerous modifications to the examples listed below without departing from the scope of this invention.

### BRIEF DESCRIPTION OF THE DRAWINGS

FIG. 1 depicts a graph of the % of proliferating cells measured 8 hours after treatment with various time periods of MW energy. Endometrial (EM Che-1) cells are compared to normal endometrial cells and skin fibroblasts.

FIG. 2 depicts a graph of the % of proliferating cells measured 72 hours after treatment with various time periods of MW energy. Again, endometrial (EM Che-1) cells are compared to normal endometrial cells and skin fibroblasts. The percentage of proliferating cells present after a 3 or more second treatment is very low, indicating this 3-5 second treatment suffices to inhibit proliferation under the conditions.

FIGS. 3, 4 and 5 show cultured cells after control treatment, 2 second MW treatment (2 s) and 5 second MW treatment (5 s).

FIGS. 6, 7 and 8 show cultured cells after control treatment, 2 second MW treatment (2 s) and 5 second MW treatment (5 s).

FIGS. 9A and 9B show the colony-forming capacity in control (9A) versus MW treated (9B) cells.

FIG. 10 shows gel electrophoresis separated genomic DNA from cells of control treatment, cells treated with MW for 1 sec (1 s), 2 sec (2 s), and 3 sec (3 s). Marker DNA (M) is shown at left. A marked degradation of the DNA can be seen in the 2 s and 3 s bands in particular.

### DETAILED DESCRIPTION OF EXEMPLARY EMBODIMENTS

In one aspect the invention involves the use of RF/MW energy on various cell types, including edometriotic and fibroblast cells. The endometriosis cell line EM.Che-1, normal endometrial stromal cells, and human skin fibroblasts are cultured in DMEM medium with 10% FCS, L-glutamine and antibiotics. For RF/MW treatment, a Nymax device (device as described in pending application WO/2008/028980 or US 2008/0065059) is used for cell treatments. The cells are grown in 96 micro-well plates and exposed to desired energy field (2450 MHz) for various time periods. Progression of cell growth over hours/days, colony-forming capacity over time, and the occurrence of DNA fragmentation are evaluated. Various methods can be used to measure these characteristics or effects in treated cells, including BrDU incorporation assays, tri-dimensional cell growth procedure in MATRIGEL membranes, and by electrophoresis of cellular DNA.

After treatment, the growth curve of the treated EM.Che-1, stromal cells and skin fibroblasts after being cultured for 3 h, 24 h and 49 hours, shows a significant decrease in the proliferation rate. The growth inhibition of the EM.Che-1 cells was stronger compared with that of the stromal cells and skin fibroblasts. In addition, colony-forming capacity of EM.Che-1 cells decreased by 42%±3 after treatment (3 sec.), which is evaluated approximately 8 days after treatment and compared to controls.

The treatment methods described here can be incorporated in numerous human and animal treatment regimens, including the treatment of endometriosis as described in Chapron, et al. (Hum Reproduct. 14: 329-332 (1999)), and the treatment of skin blemishes and wrinkles as described in WO/2008/028980 or US 2008/0065059. Other conditions involving the selective destruction of proliferating cells, such a cancers, can also benefit from the methods described here.

### EXAMPLES

### Endometriotic Cell Culture Model (1)

A human cell line, designated Em Che-1, is obtained from the biopsy of the peritoneal nodule from a 35 year old patient. The cells are grown in DMEM+10% FCS+L-glutamine+antibiotics over a basement membrane substrate (MATRIGEL™ Basement Membrane Matrix; BD Biosciences). The cells exhibit a stromal-like, adherent cell morphology and have the capacity to form colonies in matrix. The cells resemble an aberrant karyotype, but have consistent expression of cytokeratin 8, 9, 18 marker proteins, estrogen receptor (ER), and progesterone receptor (PR). The cells are shown in FIGS. 4 and 5.

### Endometriotic Cell Culture Model (2)

Cultured autologous endometrial stromal cells (shown in FIGS. 7 and 8) are maintained in DMEM medium supplemented with 10% FCS, L-glutamine, and antibiotics.

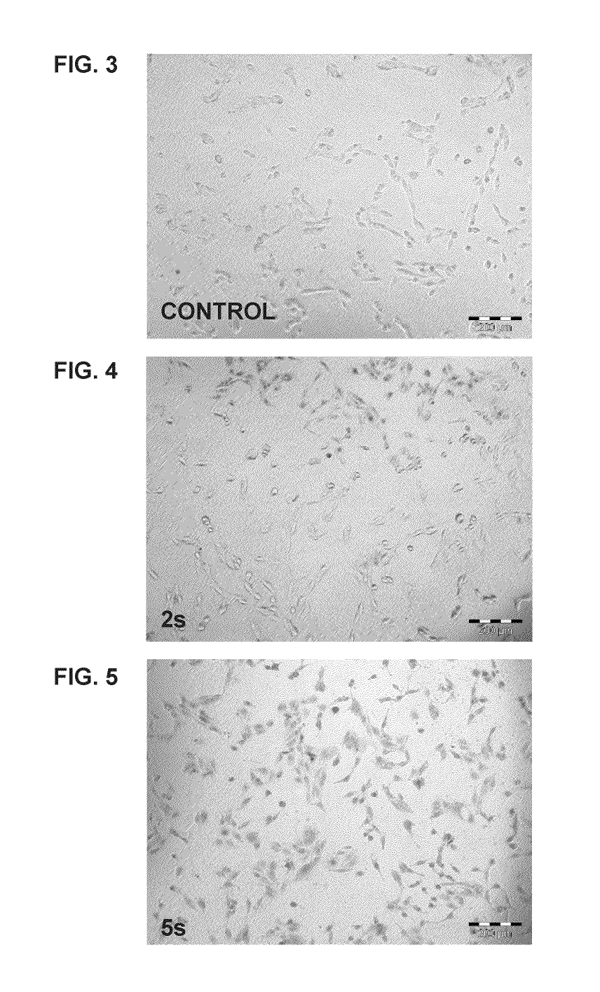

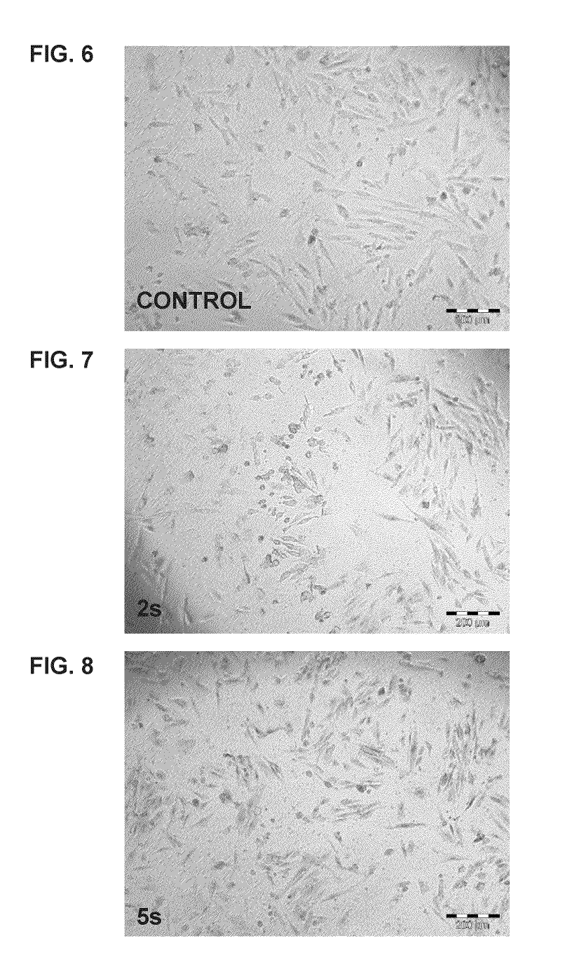

Human skin fibroblast cells (FIGS. 3 and 6) can also be used in MW treatments and used for controls as compared to the proliferating endometrial cells.

A cell proliferation rate can be established using assays known in the art, for example the TACS™ XTT cell proliferation assay (Trevigen, Inc). Similarly, colony-forming capacity can be determined by culturing in MATRIGEL™ (BD Biosciences), and cellular DNA fragmentation can be evaluated using standard gel electrophoresis of isolated genomic DNA.

RF/MW Exposure System

An RF/MW treatment device (as described in pending application WO/2008/028980 or US 2008/0065059) is used to allow very accurate energy transfer through a bipolar, handheld probe. Cells can be maintained to sub-confluence in 96-well plates, exposed to 2.45 GHz with average power of 10 W for various times, for example ranging from about 1 sec to about 12.5 sec. Typical energy delivery can be between about 5 and about 125 Joules.

Results

Effect of RF/MW on Cell Growth

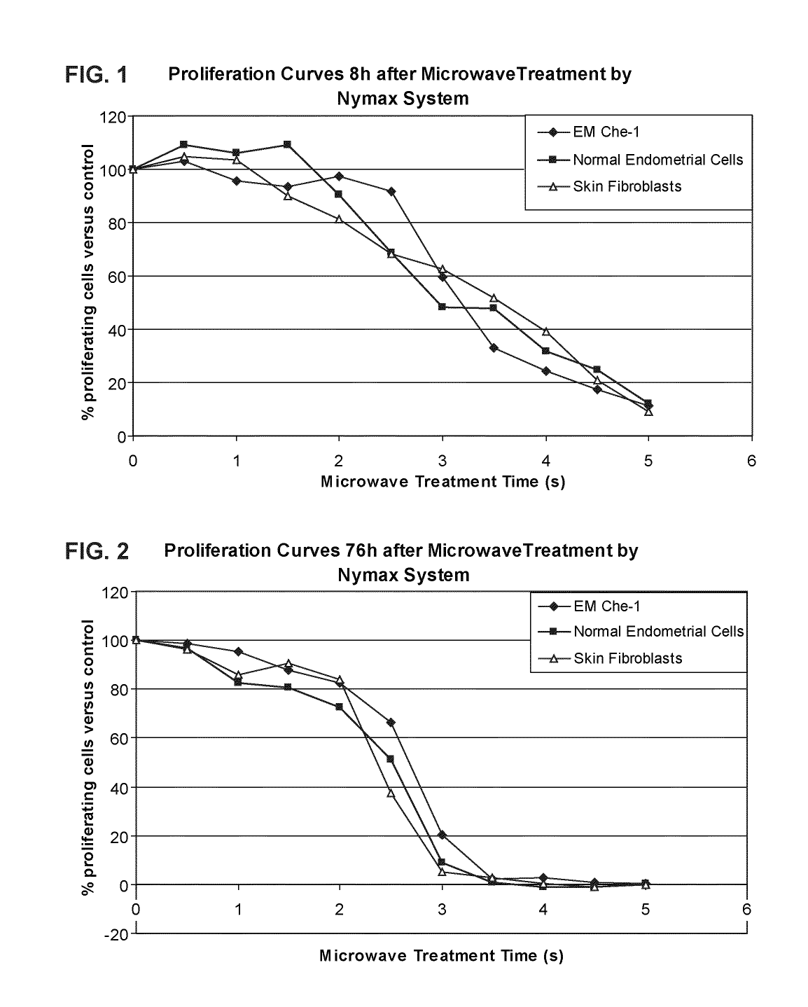

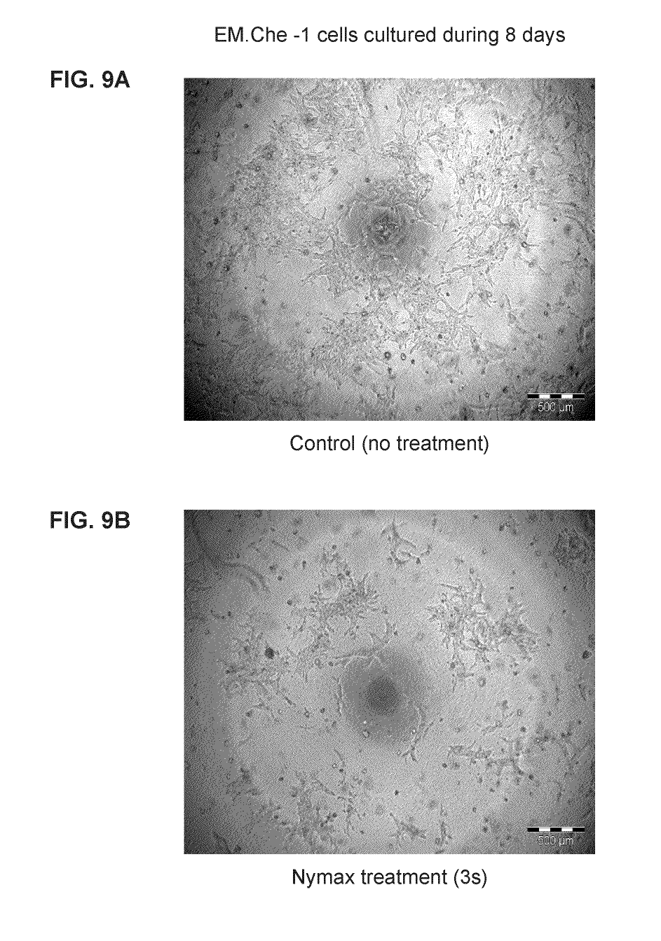

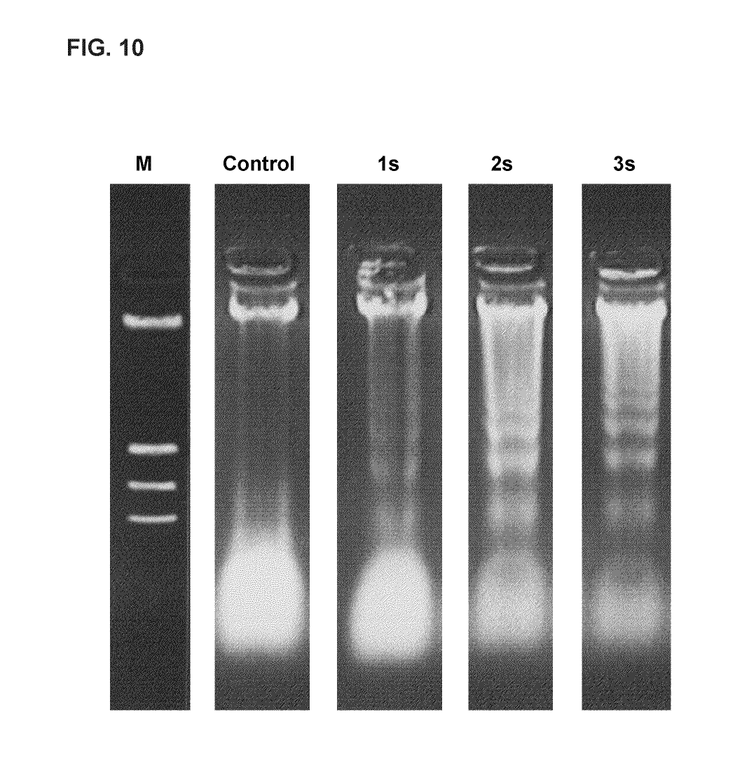

After exposure to RF for different times (0 to 5 sec), the growth curves (after 8 hrs and 76 hrs, FIGS. 1 and 2) of EM.Che-1 endometriotic cells, autologous endometrial stromal cells, and human skin fibroblasts showed a significant decrease of the proliferation rate. The photomicrographs of the cells in FIGS. 3-8 show the same results. Colony-forming capacity is compared in the photomicrographs of FIGS. 9A and 9B, where the MW treated endometriotic cells (9B) show a marked reduction in number of colonies 8 days after the 25 to 125 Joule treatment as compared to the untreated endometriotic cells. Genomic cell DNA degradation can be measured by isolating genomic DNA using conventional methods and separating the DNA by gel electrophoresis. Typical results are depicted in the comparison of bands in the gel of FIG. 10.

Laparoscopic Treatment

Laparoscopic treatment devices, such as those available in the art (see, for example, Karl Storz, Thubingen, Germany) can be used to visualize the effect on cells during and after treatment to monitor optimal treatment regimens. The device can be used in conjunction with the RF/MW treatment device noted above. A fluorescence probe to indicate endometriotic cells can also be added to the treatment, so that specific treatment sites can be visualized.

The examples presented above and the contents of the application define and describe examples of only some of the many methods, treatment regimens, human treatments, and cell measurement processes encompassed by the invention. Additional products, devices, and methods can be produced or used according to the invention. None of the examples and no part of the description should be taken as a limitation on the scope of the invention as a whole or of the meaning of the following claims.

What is claimed is:

1. A method of treating cells to prevent cell proliferation comprising treating the cells with RF/MW energy at one of more of: about 2.45 GHz with average power of 10 W for a desired period of time between 1 sec and 15 sec; or between about 5 and about 125 Joules, and measuring one or more of: decrease in cell growth or proliferation over time; capacity for colony-forming; or genomic DNA degradation.

2. A method for determining a cell growth inhibiting RF/MW treatment, comprising applying RF/MW energy from a bipolar device to the cells, and measuring one or more of: decrease in cell growth or proliferation over time; capacity for colony-forming; or genomic DNA degradation.

3. A method of treating endometriosis comprising providing a RF/MW emitting device and delivering from about 5 to about 125 Joules, whereby the cells capacity for growth or colony formation is decreased.

4. The method of claim 3, wherein the emitting device is inserted into the body using minimally invasive techniques.

5. A MW emitting device for treating endometriosis or cell proliferating disease, comprising a handheld bipolar tip for emitting MW incorporated into a laparoscopic surgical wand, the device capable of delivering about 25 to about 125 Joules, or about 2.54 GHz at about 10 W for 1 to 15 seconds.

6. The device of claim 5, configured for the laparoscopic treatment of endometriosis in humans.

7. A method as claimed in claim 1 for the treatment of endometriosis in a patient in need thereof, wherein a device to deliver RF/MW energy is configured for laparoscopic surgery in a human, and whereby the treatment results in cell damage to proliferating cells associated with the endometriosis.

8. A method as in claim 1, further comprising treating cells or tissue with a fluorescent or autofluorescent compound, whereby the treatment area or cells are indicated by fluorescence.

9. A method as in claim 2, further comprising treating cells or tissue with a fluorescent or autofluorescent compound, whereby the treatment area or cells are indicated by fluorescence.

10. A method as in claim 3, further comprising treating cells or tissue with a fluorescent or autofluorescent compound, whereby the treatment area or cells are indicated by fluorescence.

11. A method as in claim 4, further comprising treating cells or tissue with a fluorescent or autofluorescent compound, whereby the treatment area or cells are indicated by fluorescence.

12. A method as in claim 7, further comprising treating cells or tissue with a fluorescent or autofluorescent compound, whereby the treatment area or cells are indicated by fluorescence.
